(19) United States
(12) Patent Application Publication
(10) Pub. No.: US 2010/0198066 A1
(43) Pub. Date: Aug. 5, 2010

(54) Method and Device for Obtaining Tissue Samples

(76) Inventor: James W. Voegele, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/757,273

(22) Filed: Apr. 9, 2010

Related U.S. Application Data
(63) Continuation-in-part of application No. 10/800,339, filed on Mar. 12, 2004.
(60) Provisional application No. 61/236,972, filed on Aug. 26, 2009.

(51) Int. Cl. A61B 8/00; A61B 10/02; A61B 18/18

(52) U.S. Cl. 600/439; 600/567; 606/33

(57) ABSTRACT

A method for obtaining one or more tissue samples, such as from the prostate, is provided. The method can include use of a handheld, vacuum assisted, single insertion multiple sample biopsy device to remove two spaced apart volumes of prostate tissue, one on either side of the urethra. A sleeve with multiple electrodes can be received on the biopsy device or otherwise associated with the biopsy device, to provide RF energy to the biopsy site and to cauterize tissue surfaces from which the tissue samples have been taken.

This patent application claims priority to provisional application Ser. No. 61/236,972 filed Aug. 26, 2009. This application also claims priority as a continuation in part to U.S. patent application Ser. No. 10/800,339 filed Mar. 12, 2004.

### CROSS REFERENCE TO RELATED APPLICATION

The present application incorporates by reference provisional application 61/236,972 filed Aug. 26, 2009, and U.S. patent application Ser. No. 10/800,339, Electrode Sleeve For Biopsy Device, filed in the name of James Voegele, and published as US2005/0203441A1.

### FIELD OF THE INVENTION

The present invention is related generally to devices and methods for the collection of soft tissue. More particularly, it relates to a device and a method which can be employed in treating Benign Prostate Hypertrophy (BPH).

### BACKGROUND OF THE INVENTION

The diagnosis and treatment of patients with cancerous tumors, pre-malignant conditions, and other disorders has long been an area of intense investigation. Non-invasive methods for examining tissue include palpation, X-ray, MRI, CT, and ultrasound imaging. When the physician suspects that a tissue may contain cancerous cells, a biopsy may be done using either an open procedure or a percutaneous procedure. For an open procedure, a scalpel is used by the surgeon to create a large incision in the tissue in order to provide direct viewing and access to the tissue mass of interest. The entire mass (excisional biopsy) or a part of the mass (incisional biopsy) may then be removed. For a percutaneous biopsy, a needle-like instrument is used through a very small incision to access the tissue mass of interest and to obtain a tissue sample for later examination and analysis. The advantages of the percutaneous method as compared to the open method may be significant and may include: less recovery time for the patient, less pain, less surgical time, lower cost, and less disfigurement of the patient's anatomy. Use of the percutaneous method in combination with imaging devices such as X-ray and ultrasound has resulted in highly reliable diagnoses and treatments.

Generally there are two ways to percutaneously obtain a portion of tissue from within the body, by aspiration or by core sampling. Aspiration of the tissue through a fine needle requires the tissue to be fragmented into pieces small enough to be withdrawn in a fluid medium. The method is less intrusive than other known sampling techniques but one can only examine cells in the liquid (cytology) and not the cells and the structure (pathology). In core biopsy, a core or fragment of tissue is obtained for histologic examination which may be done via a frozen or paraffin section.

The type of biopsy used depends mainly on various factors present in the patient, and no single procedure is ideal for all cases. Core biopsy, however, is very useful in a number of conditions and is widely used by physicians.

The biopsy device used should be lightweight, maneuverable, and handheld so that the surgeon may have the option to perform the biopsy procedure in combination with an ultrasound imaging device. In addition, the biopsy device should perform a biopsy procedure with fewer steps decreasing the overall time of the procedure.

In some cases it is desirable that the surgeon be able to easily steer the penetrating tip of the handheld device towards the desired tissue to be sampled. It may further be desired that the surgeon have tactile feedback as the tissue is proved by the penetrating tip of the device, to provide the surgeon with clues regarding the disease state of the tissue encountered.

Bleeding of tissue at the knife tip and around the piercer of the biopsy device may occur with current biopsy devices. Past devices have alleviated bleeding by placing an electrode through a cannula, removing the electrode, and then placing suction and cutting devices through the cannula to take the biopsy. Applicants have recognized a need for an instrument that cauterizes tissue and takes a biopsy core sample in a single step without removal and reinsertion of a device. To accomplish the single-insertion goal, applicants have further recognized a need for electrodes disposed on the outer surface of the piercer behind the tip. To enable removability of the electrodes, applicants have recognized the need for location of the electrodes on a sleeve that can be removably placed on the piercer. Because of the possibility of bleeding caused by the knife tip, applicants have recognized a need for the knife tip allowing for cauterization at the knife tip. Applicants have further recognized a need for a switching relay to alternately energize electrodes and knife tip to effectively cauterize different areas of the penetration site.

The following patent documents are incorporated herein by reference in their entirety: U.S. Pat. No. 6,273,862 issued Aug. 14, 2001; U.S. Pat. No. 6,231,522 issued May 15, 2001; U.S. Pat. No. 6,228,055 issued May 8, 2001; U.S. Pat. No. 6,120,462 issued Sep. 19, 2000; U.S. Pat. No. 6,086,544 issued Jul. 11, 2000; U.S. Pat. No. 6,077,230 issued Jun. 20, 2000; U.S. Pat. No. 6,017,316 issued Jan. 25, 2000; U.S. Pat. No. 6,007,497 issued Dec. 28, 1999; U.S. Pat. No. 5,980,469 issued Nov. 9, 1999; U.S. Pat. No. 5,964,716 issued Oct. 12, 1999; U.S. Pat. No. 5,928,164 issued Jul. 27, 1999; U.S. Pat. No. 5,775,333 issued Jul. 7, 1998; U.S. Pat. No. 5,769,086 issued Jun. 23, 1998; U.S. Pat. No. 5,649,547 issued Jul. 22, 1997; U.S. Pat. No. 5,526,822 issued Jun. 18, 1996; US 2003/0199785 published Oct. 23, 2003; US 2003/0199754 published Oct. 23, 2003; US 2003/0199754 published Oct. 23, 2003.

### SUMMARY OF THE INVENTION

In one embodiment, the present invention provides a method for obtaining one or more tissue samples, such as from the prostate. The method can include use of a handheld, vacuum assisted, single insertion multiple sample biopsy device to remove spaced apart volumes of prostate tissue, such as one on either side of the urethra. The method may also employ a sleeve with multiple electrodes received on the biopsy device or otherwise associated with the biopsy device.

### BRIEF DESCRIPTION OF THE FIGURES

FIG. 1 is an isometric, expanded view of a probe assembly with the electrode sleeve, as disclosed in US2005/0203441A1.

FIG. 2 is an isometric view of the electrode sleeve.

FIG. 3 is an isometric view of the probe assembly with the electrode sleeve attached.

FIG. 4 is a schematic diagram of an interface relay board setup.

FIG. 5 is an isometric view of a biopsy instrument including a hand piece for the collection of soft tissue.

FIG. 6 is an isometric view of the probe assembly with the left handle shell removed to reveal internal components.

FIG. 7 is an isometric view of the handpiece showing the probe assembly prior to attachment to a holster.

FIG. 8 is an isometric view of an alternate embodiment of the electrode sleeve as disclosed in US2005/0203441A1.

FIG. 9 is an isometric view of an alternate embodiment of an exploded view of the probe assembly with the electrode sleeve slightly distal to the piercer.

FIG. 10 is an isometric view of an alternate embodiment of the probe assembly with the electrode sleeve attached.

FIG. 11 is an isometric view of an alternate embodiment of the probe assembly with the electrode sleeve slightly distal to handle 43.

FIG. 11a is an isometric view of a breakout view of the contact in the groove on the underside of the handle.

FIG. 11b is an isometric view of a breakout view of the contacts on the connector on the proximal end of the electrode sleeve.

FIG. 12 is a cross-sectional illustration of electrodes placed on a hollow piercer with a tissue cutter disposed for translation and rotation within the piercer.

FIG. 13 is a cross-sectional illustration of electrodes placed on a hollow electrode sleeve disposed on a hollow piercer, with a tissue cutter disposed for translation and rotation within the piercer.

FIG. 14 is a schematic illustration of an ultrasound probe inserted in the rectum to assist in visualizing the prostate by ultrasound imaging.

FIG. 15 is a schematic illustration of a single insertion, multiple sample vacuum assisted biopsy device being inserted at the anal verge to obtain samples of prostate tissue under ultrasound guidance.

FIG. 15A is an enlarged schematic illustration showing a catheter having an inflatable balloon portion positioned such that a tip of the catheter is disposed in the bladder and the inflatable portion is disposed within a portion of the urethra extending through the prostate, with the inflatable portion being at least partially inflated to help support the urethra and assist in identifying the urethra under ultrasound image guidance.

FIG. 16 is a schematic illustration of an electrode sleeve according to one embodiment of the present invention.

FIG. 17 is a schematic illustration of an electrode sleeve disposed on the piercer of a vacuum assisted biopsy device, where the piercer has distal piercing tip comprising a conductive electrode.

FIG. 18 is a schematic illustration showing a vacuum assisted biopsy device and electrode sleeve inserted into a left side of the prostate with a tissue sampling aperture facing away from the portion of the urethra extending through the prostate.

FIG. 18A is a cross-sectional schematic illustration of the portion of the prostate sampled in FIG. 18 and illustrating removal of tissue samples.

FIG. 19 is a schematic illustration showing a vacuum assisted biopsy device and electrode sleeve inserted into a right side of the prostate with tissue sampling aperture facing away from the portion of the urethra extending through the prostate.

FIG. 19A is a cross sectional schematic illustration of the portion of the prostate sampled in FIG. 19.

FIG. 20 is a schematic illustration depicting voids created within the prostate on opposite side of the urethra using the device and method illustrated in FIGS. 14-19.

FIG. 21A illustrates a generally cylindrical volume of cauterized tissue 3106A (such as tissue surrounding a volume from which a core sample 3105 is taken), the cauterized tissue being provided such as by energizing two electrodes associated with a lateral tissue sample opening through which a core sample is taken.

FIG. 21B illustrates a volume of tissue including a generally cylindrical volume of cauterized tissue, such as shown in FIG. 21A, with an end cap shaped volume of cauterized tissue 3106B, where the end cap shaped volume of tissue can be provided by alternatively energizing a distal tip or end electrode with one of two electrodes associated with a lateral tissue sample opening.

FIG. 21C illustrates a partial cross-section as taken along the lines 21C-21C in FIG. 21B and illustrating cauterized tissue 3106A and 3106B in prostate 3100 adjacent a volume from which a core sample 3105 has been taken.

### DETAILED DESCRIPTION OF THE INVENTION

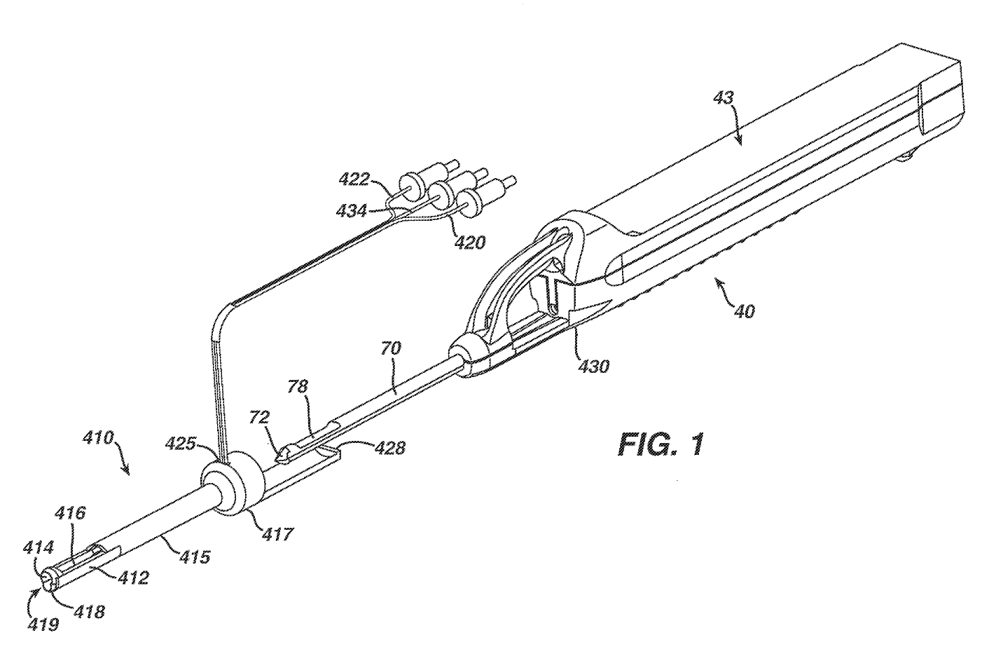

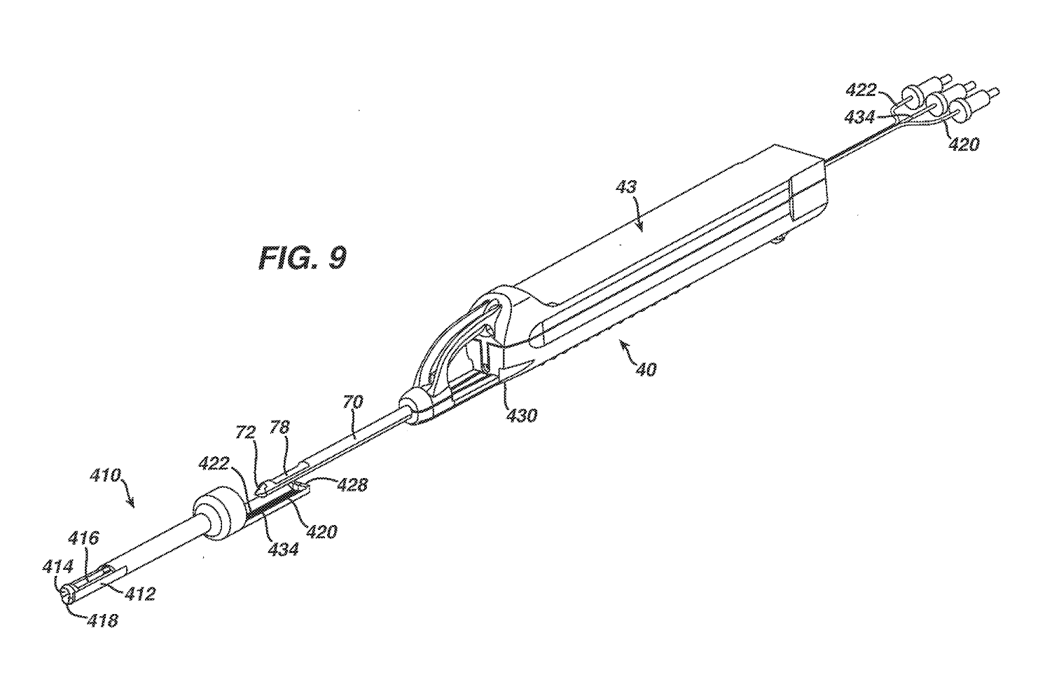

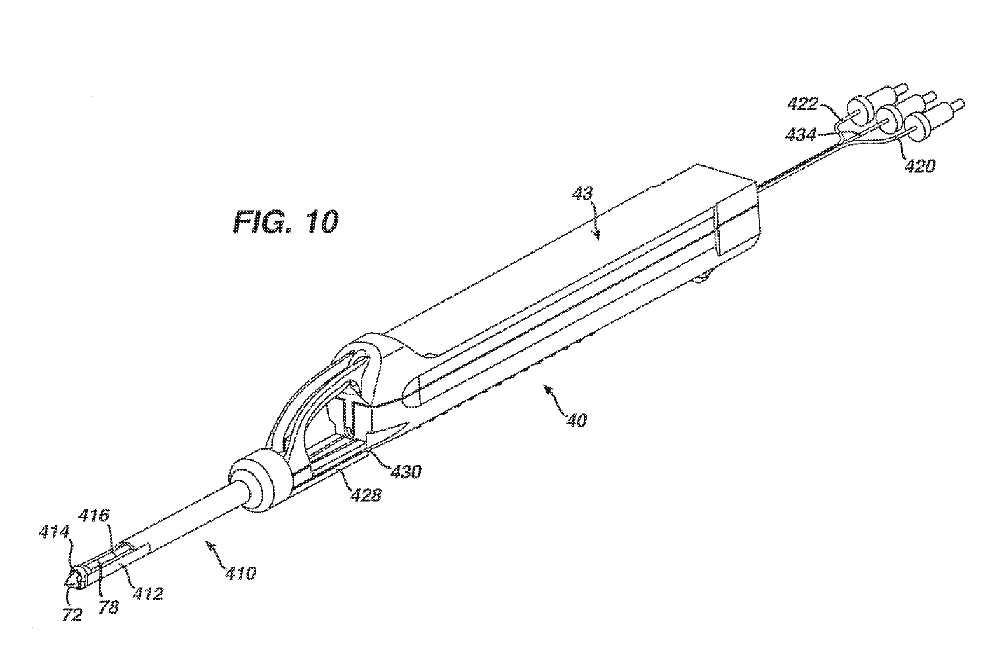

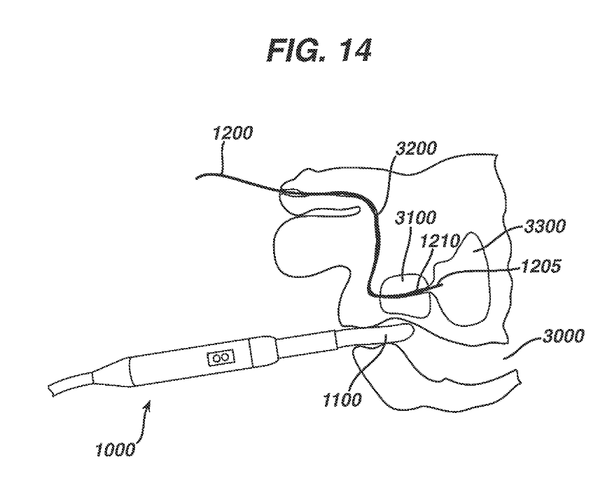

FIGS. 1-13 illustrate embodiments of a device described in US2005/0203441A1. FIGS. 14-20 illustrate the device and method of the present invention.

FIG. 1 discloses an exploded view of a probe assembly 40 and a sleeve 410. Probe assembly 40 may be a probe assembly as disclosed in U.S. Pat. No. 6,273,862, “Surgical Device for the Collection of Soft Tissue,” the entire contents of which are hereby incorporated herein by reference. A suitable probe assembly 40 is a part of the MAMMOTOME® breast biopsy instrument, available from Ethicon Endo-Surgery, Cincinnati, Ohio. Probe assembly 40 can include a hollow cannular piercer 70 extending distally from a hollow handle 43, a distal piercing knife tip 72 at the distal end of piercer 70, and a groove 430 on the underside of handle 43. Sleeve 410 can include a first electrode 412, a second electrode 414, a tissue receiving window 416, an electrode gap 418, a connector 428, a first wire 420 attached to electrode 412, a second wire 422 attached to electrode 414, and a third wire 434. Sleeve 410 is sized to slide over piercer 70. Sleeve 410 is placed over piercer 70 by sliding sleeve 410 proximally towards handle 43. The sleeve 410 can include a connector 428 for releasably connecting the sleeve 410 to the probe assembly 40. Connector 428 extends proximally from sleeve 40 and joins with groove 430 on the underside of handle 43 to connect sleeve 410 to handle 43. When sleeve 410 is connected to probe assembly 40, sleeve 410 abuts the distal end of handle 43, and knife tip 72 can protrude from an open distal end of sleeve 410 when sleeve 410 is connected to handle 43.

Electrode geometry may be as disclosed in World Patent Application No. 02/078557 to Gary Long filed on 29 Mar. 2002, and incorporated herein by reference. The size, shape, and relative position of electrodes 412 and 414 are established by Ablation Index,

### I, and:

I=P/d

### Where:

P is the perimeter of electrodes 412 and 414 and
d is the separation between adjacent edges of electrodes 412 and 414 on the bottom of the sleeve 410, the separation d corresponding to electrode gap 418 in the Figures.

In the embodiment of the invention having generally rectangular electrodes:

I=2(w+L)/d

Where:
w is the width of electrodes 412 and 414 and
L is the length of electrodes 412 and 414 measured parallel to the long axis of the sleeve 410.

Suitable ablation indices can be provided wherein: the separation d can be between about 1 mm and about 3 mm: L can be between about 20 mm and about 40 mm: and w can be between about 3 mm and 8 mm. In particular, d can be less than or equal to about 3 mm. More particularly, electrode size and spacing of d equal to 2 mm, L equal to 30 mm, and w equal to 5 mm can be used to provide an Ablation Index I=35. In another specific embodiment, electrode size and spacing of d equal to 3 mm, L equal to 30.4 mm, and w equal to 5.08 mm can be used to provide an Ablation Index I=23.

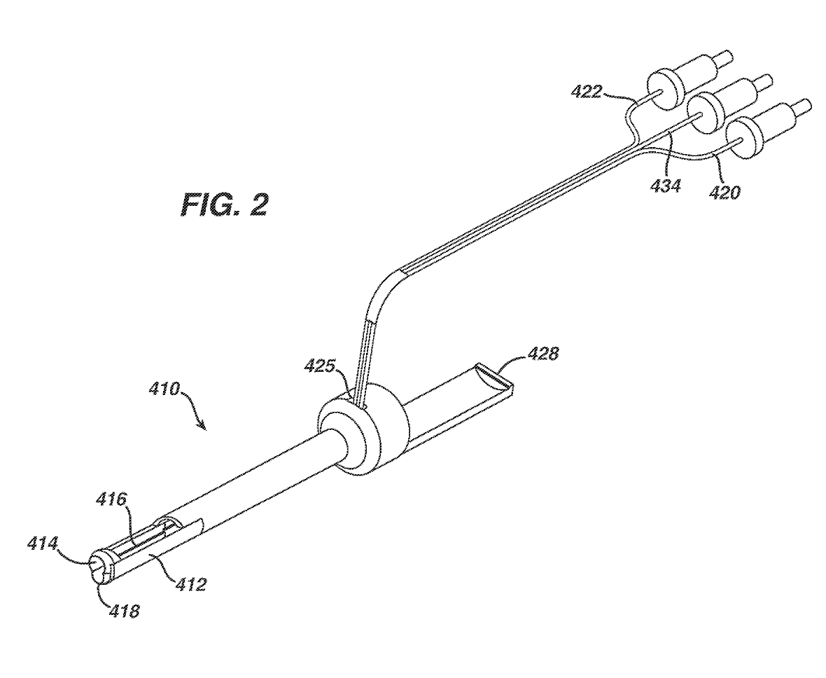

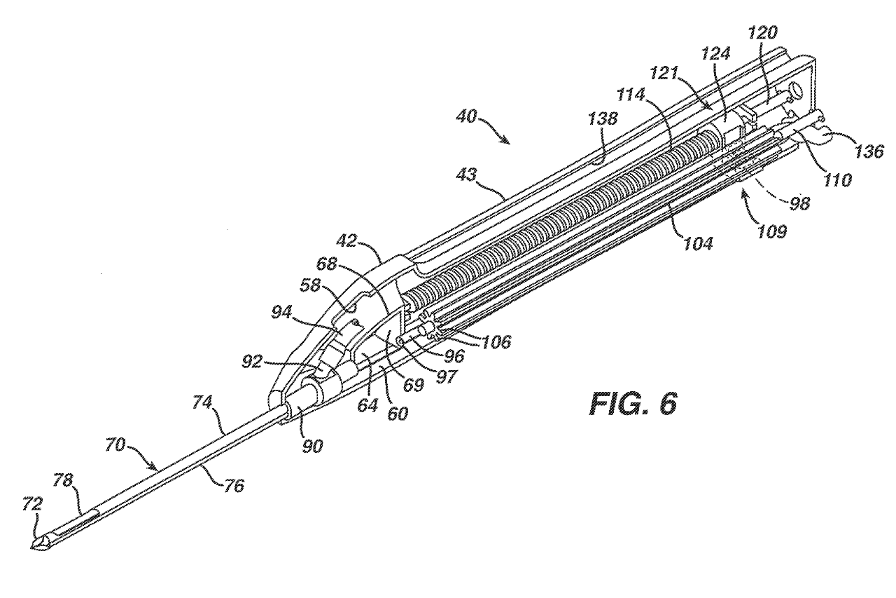

FIG. 2 depicts features of electrode sleeve 410. Electrode sleeve 410 has dimensions allowing electrode sleeve 410 to slide proximally over piercer 70 towards handle 43. Piercer 70 can have dimensions of an 8 gauge (0.165 inches), 11 gauge (0.120 inches), or a 14 gauge (0.083 inches) needle and the length of piercer 70 can be approximately 3.27 inches. A cutter 96 (FIG. 6) translates and rotates such that a distal end of cutter 96 translates and rotates within piercer 70 relative to lateral tissue receiving port 78 to sever tissue samples received in opening 78.

Sleeve 410 can include an elongated, hollow body portion 415 extending distally from a shoulder portion 417. Body portion 415 can include a generally rectangular window 416 and an end opening 419 at the distal end of body portion 415. Window 416 can be spaced proximally from the open distal end opening 419, near the distal end opening 419. Window 416 provides an opening in sleeve 410 which can be aligned with the tissue receiving port 78 of piercer 70. Window 416 allows port 78 to receive tissue extracted from the surgical patient. It is desirable for window 416 to be aligned with port 78 when performing the biopsy. Electrodes 412 and 414 can be positioned alongside of window 416 on the exterior surface sleeve 410, with electrode gap 418 separating electrodes 412 and 414. Electrode gap 418 corresponds to the separation d between electrode 412 and electrode 414, and electrode gap 418 corresponds to the spacing between electrodes 412 and 414 opposite window 416.

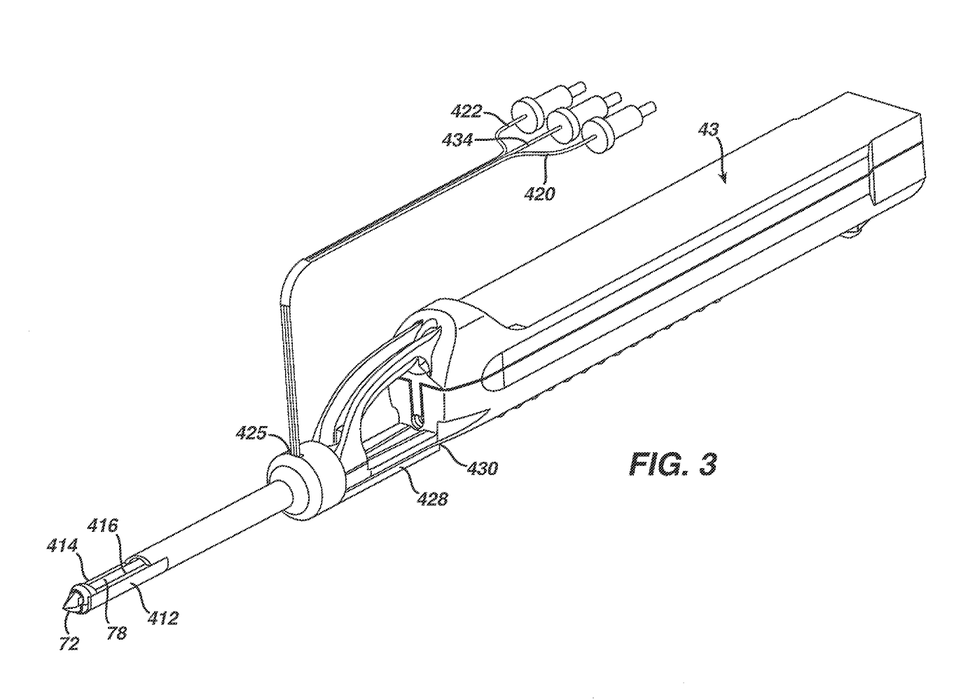

FIG. 3 depicts electrode sleeve 410 attached to probe assembly 40. Connector 428 is joined with groove 430 on the underside of probe assembly 40.

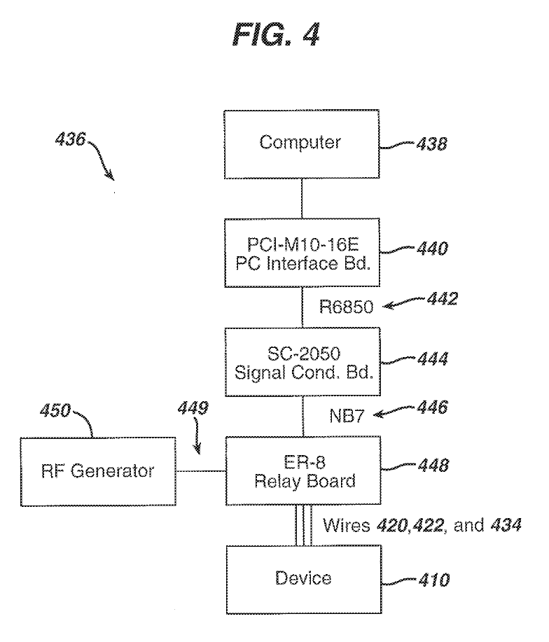

Wires 420 and 422 electrically connect to electrodes 412 and 414, respectively. A third wire 434 can be provided. The distal portion of wire 434 can be located in the interior of electrode sleeve 410, and the plastic covering or other insulation can be removed from the distal portion of wire 434 so that wire 434 can be in electrical contact with piercer 70 and/or the knife 72 of piercer 70, while the proximal portion of wire 434 extending proximally from electrode sleeve 410 can comprise a plastic covering or other insulating covering. Wires 434,420 and 422 can extend from electrode sleeve 410 at a wire opening 425. The proximal ends of wires 420, 422, and 434 can be electrically connected to an interface relay board as shown schematically in FIG. 4.

FIG. 4 schematically depicts in block diagram form components associated with operation of sleeve 410, the interconnected components indicated by reference number 436 in FIG. 4. The components comprise a computer 438, a PC interface board 440, a cable 442, a Cable adaptor 444, a cable 446, a relay board 448, wires 420, 422, and 434, RF generator 450, a cable 449, and electrode sleeve 410. Computer 438 is connected to PC interface board 440. PC interface board 440 is connected to cable adaptor 444 by cable 442 Cable 446 connects cable adaptor 444 to relay board 448. RF generator 450 is connected to relay board 448 via a two-gauge cable 449. Wires 420, 422, and 434 connect to relay board 448 and to electrode sleeve 410.

PC interface board 440 is a multi-function component of assembly 436. For the specific embodiment disclosed, only the switching function of this multi-function component is used. PC interface board 440 switches electronic components on and off. Three of the eight switches contained in PC interface board 440 are used. Computer 438 can be programmed to control PC interface board 440 to indicate which switches of the three are on and which switches are off. Cable adaptor 444 is used to connect cable 442 and cable 446.

Relay board 448 acts as the physical relay from RF generator 450 to wires 420, 422, and 434. Relay board 448 uses switching instruction from PC interface board 440 to relay current and voltage from RF generator 450 to the correct electrode or electrodes.

RF generator 450 creates the radio frequency current used to provide RF energy to the electrodes. Relay board 448 directs RF energy to wires 420, 422, and 434, according to instructions provided by computer 438.

Boards and wires may be purchased as catalog components from National Instruments in Austin, Tex. Suitable part numbers are: for PC interface board 440/part no. PCI-M10-16E; for cable 442/part no. R6850; for cable adaptor 444/part no. SC-2050; for cable 446/part no. NB7; for Relay Board 448/part no. ER-8. A suitable RF generator 450 is a Valley Lab Force 2 generator available from Valleylab located in Boulder, Colo. Suitable software for providing control of RF energy to the electrodes is LabView Software v.6.0, available from National Instruments in Austin, Tex.

Once programmed and initiated, the components shown in FIG. 4 can be used to provide a switching circuit to alternate charge to either electrodes 412 and 414, electrode 412 and piercer 70, or electrode 414 and piercer 70. Wires 420 and 422 supply current to electrodes 412 and 414 respectively, while wire 434 supplies current to tip 72 through piercer 70. When a metallic or otherwise conducting piercer 70 is employed, distal piercing knife tip 72 is energized when wire 434 is energized. Alternatively, wire 434 can be configured to make direct contact with a conductive tip 72.

In one embodiment, the components indicated by numeral 436 can be employed to alternately charge as a pair electrode 412 and electrode 414, then electrode 412 and knife tip 72, then electrode 414 and knife tip 72.

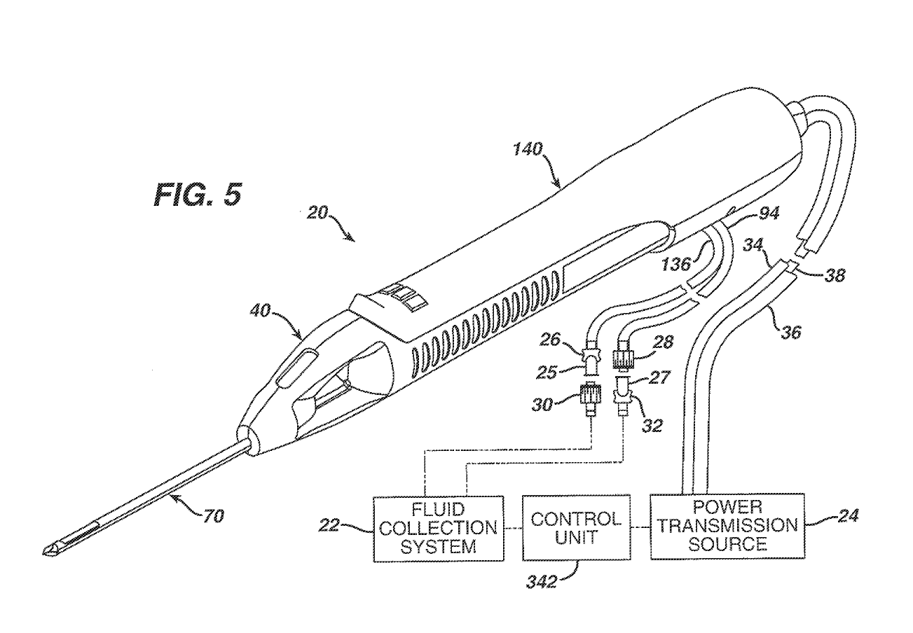

FIG. 5 depicts an embodiment of a biopsy instrument comprising a probe assembly 40, a holster 140, a fluid collection system 22, a control unit 342, and a power transmission source 24 as disclosed in U.S. Pat. No. 6,273,862, “Surgical Device for the Collection of Soft Tissue.” The probe assembly 40 is detachably connected to the holster 140. Together they constitute a lightweight, ergonomically shaped, hand manipulatable portion referred to as a handpiece 20. The probe assembly 40 includes a piercer 70 extending distally from a hollow handle 43. The probe assembly 40 is fluidly connected to the fluid collection system 22 by a first vacuum tube 94 and a second vacuum tube 136. The first and second vacuum tubes are detachably connected to the fluid collection system 22 by a first connector 27 and a second connector 25, respectively. The first connector has a male portion 32 and a female portion 28 attached to the first vacuum tube 94. The second connector 25 has a female portion 30 and a male portion 26 attached to the second vacuum tube 136. The connector portions, 26, 28, 30, and 32, are attached in this manner to prevent the accidental switching of the first and second tubes, 136 and 94, to the fluid collection system 22. The holster 140 includes a first rotatable shaft 34, a second rotatable shaft 36, and a control cord 38. The first and second rotatable shafts, 34 and 36, are preferably flexible so that the operator may easily manipulate the handpiece 20 with one hand. The control cord 38 operatively connects the handpiece 20 to the power transmission source 24 and control unit 342.

Referring to FIG. 6, an isometric view of the probe assembly 40 with the left portion of handle shell 44 removed reveals the placement of the components. Part of the first vacuum tube 94 has also been removed for clarity. The carriage 124 is shown in the fully retracted position so that the cutter 96 is also at the fully retracted, or first position. The cutter blade 97 is slightly distal to the vertical wall 69 on the handle 43. The foot of the carriage 124 is adapted to slide along a carriage guide surface 60 on the inside bottom of the hollow handle 43. As shown, a cutter axial transmission 121 includes the carriage 124, the screw 114, and the screw shaft 120. A cutter rotational transmission 109 includes the drive gear 104, the cutter gear 98, and the gear shaft 110.

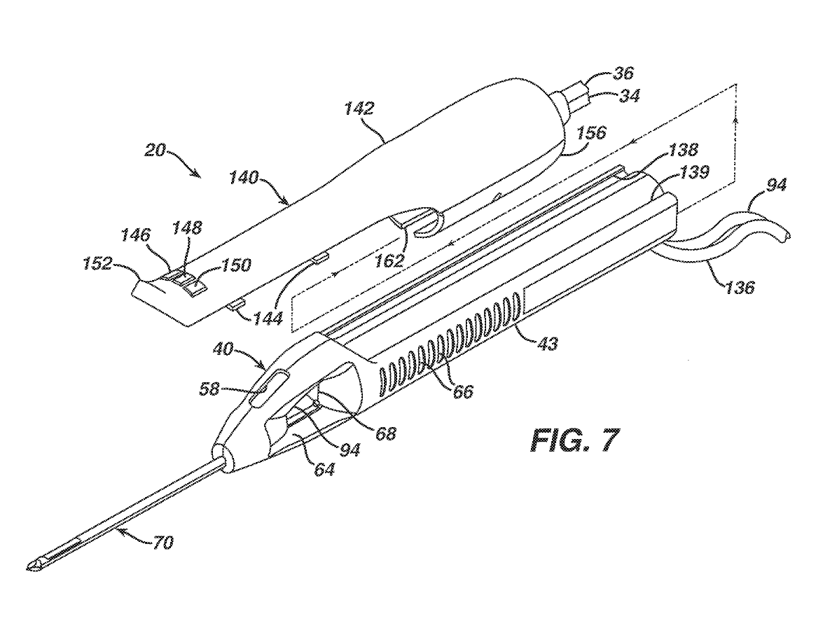

Referring to FIG. 7, the holster 140 and the probe assembly 40 are shown separated. A pair of tabs 144 project laterally from each side of a holster upper shell 142, and insert into right and left undercut ledges, 138 and 139 respectively, of the hollow handle 43 of the probe assembly 40. A plurality of indentations 66 are provided on the handle 43 to improve the operator's grip on the instrument. A tube slot 162 in the lower shell 156 of the holster L40 provides clearance for first and second vacuum tubes, 94 and 136. A first switch 146, a second switch 148, and a third switch 150 are mounted in the distal portion of the holster 140 so that the physician can operate the handpiece 20 with a single hand while having the other hand free to operate an ultrasonic imaging device or the like. The switches 146, 148, and 150 are provided to operate the power transmission source 24 and the fluid collection system 22 in conjunction with the control unit 342. A ridge 152 on the distal end of the holster 140 is provided to assist the operator in grasping the handpiece 20 and in operating the switches 146, 148, and 150. The ridge 152 further provides the operator with a tactile reference as to where to properly grasp the handpiece 20.

Still referring to FIG. 7, the probe assembly 40 includes a window 58 so that a portion of the first vacuum tube 94 may be viewed. The first and second vacuum tubes, 94 and 136, can be made from a flexible, transparent, or translucent material, such as silicone tubing. This enables visualization of the material flowing through the tubes. By having the window 58 in the probe assembly 40, the operator can see the flow in the first tube 94 without needing to look away from the tissue into which the piercer 70 is inserted. A transverse opening 68 is provided in the distal end of the hollow handle 43 which allows access from either side to a tissue sampling surface 64. The tissue extracted from the surgical patient is retrieved by the operator or an assistant from the tissue sampling surface.

Prior to obtaining a biopsy sample, the electrode sleeve 410 can positioned over piercer 70 with window 416 aligned with tissue receiving port 78, and wires 422, 420, and 434 can be connected as shown in FIG. 4. Piercer 70 can be positioned in breast tissue to be biopsied. Vacuum can be provided at tissue port 78 so that soft tissue adjacent to port 78 prolapses into port 78 through window 416 when the first vacuum tube 94 is fluidly connected to the vacuum of the fluid collection system 22. The tissue pulled into port 78 is then severed by rotating and advancing cutter blade 97 and stored inside the cutter lumen of the cutter 96. Cutter 96 can then be retracted proximally to a first position so that cutter blade 97 is just distal to the tissue sampling surface 64, and a stationary tissue knockout pin can be used to push the severed tissue sample from the cutter onto the surface 64.

After one or more biopsy samples have been obtained, the operator can then coagulate the breast tissue at the sample site by energizing the electrodes 412 and 416 associated with the sleeve 410. If desired, tissue can be cauterized during insertion of the piercer 70 into tissue. For instance, the electrodes associated with sleeve 410 and/or the tip 72 can be energized during insertion of the piercer 70 to reduce bleeding at the insertion site.

In one embodiment, RF generator 450 can provide about 70 amps, while the switching generated from interface relay board setup 436 changes each electrode pair for a time of about 2500 milliseconds. The process of systematically switching charged electrodes is repeated until completion of coagulation. The surgeon can rotate the biopsy device with electrode sleeve 410 attached to align electrodes with different portions of the tissue to ensure hemostasis of the entire tissue area. Once the core biopsy sample has been retrieved and hemostasis exists in tissue, the biopsy device can be removed from the breast tissue or prepared for another core biopsy.

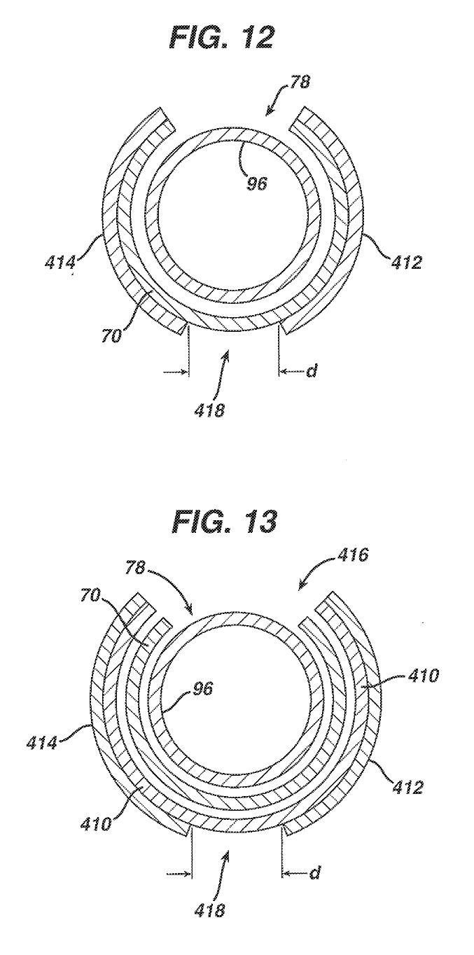

FIG. 13 provides a cross-sectional view of the electrode sleeve 410 positioned on a piercer 70, with the cross-sectional view taken perpendicular to the axis of the piercer 70 and through the tissue port 78. Electrode sleeve 410 can be formed of a suitable non metallic material, such as a plastic or polymeric material. For instance, sleeve 410 can be formed of a liquid crystal polymer available as Vectra® brand liquid crystal polymer available from Ticona Company of Germany. In FIG. 13, coagulation can be provided at gap 418 when electrodes 412 and 414 are energized. Alternatively, when current from the generator is provided to the piercer 70 and to one of the electrodes 412 or 414, coagulation can occur in tissue extending into sleeve window 416 and aligned tissue port 78 due to the conductive path through the tissue at either edge of the tissue port 78, depending on which electrode 412 or 414 is activated. Applicant has found that it is not necessary to rotate any of the components within the tissue mass (such as the piercer 70 and sleeve 410) to obtain substantially 360 degree coagulation around the piercer/sleeve. Without being limited by theory, it is believed that such 360 degree coagulation of tissue (such as breast tissue) without rotation of the piercer 70 and sleeve 410 can be accomplished because of the change of impedance through the tissue as coagulation progresses and heating of the electrodes 412 and 414. In an alternative embodiment, electrodes 412 and 414 could be attached directly to piercer 70, as shown in FIG. 12. Piercer 70 could then be formed of a non-conductive material, such as an engineering plastic. For example, Vectra® brand liquid crystal polymer, available from Ticona in Germany could be used as the piercer material. Attaching electrodes 412 and 414 to piercer 70 eliminates the need for a separate sleeve with electrodes 412 and 414 attached. The third electrode could be formed by knife tip 72 being electrically connected to handle 43. Knife tip 72 would be charged when cutter 96 contacts the proximal surface of knife tip 72 to coagulate tissue positioned generally between the knife tip 72 and the electrodes 412/414. Further, the portion of the cutter within tissue port 78, when energized along with energization of one or both of the electrodes 412/414, can provide coagulation of tissue proximal of the knife tip 72. In this embodiment, wires 420, 422, and 434 can be connected as previously disclosed.

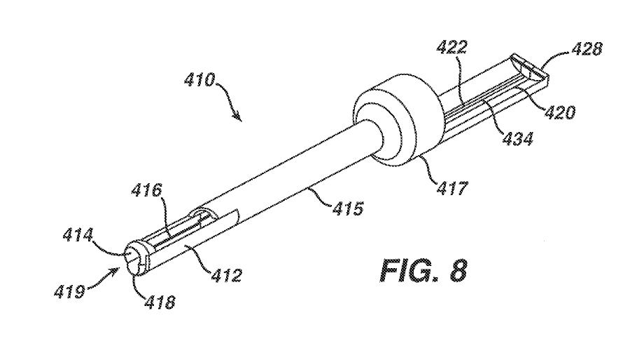

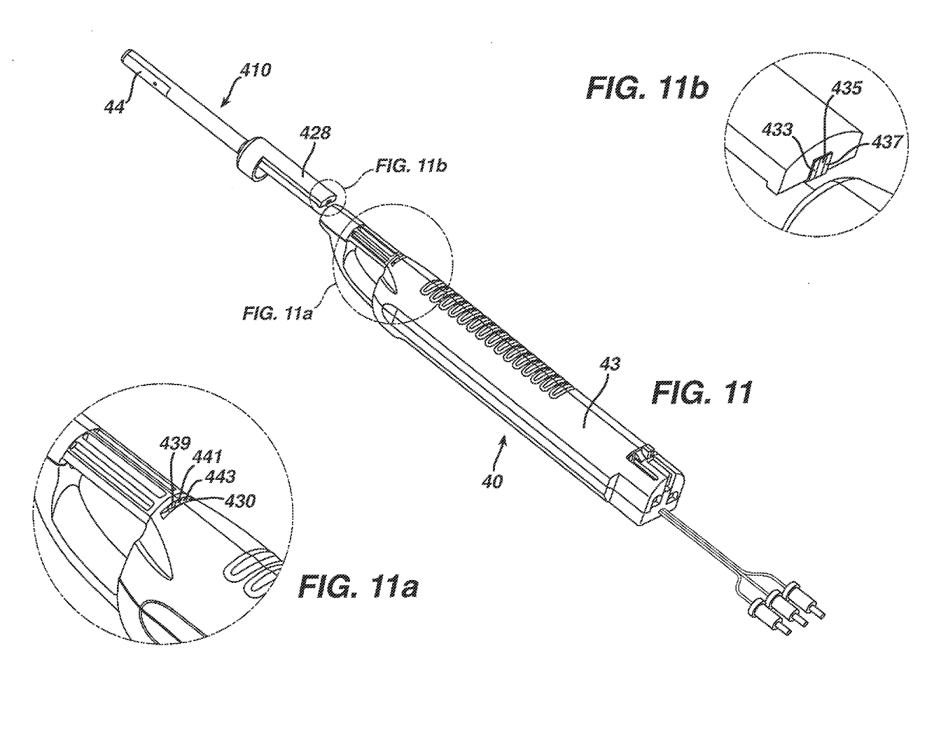

Another embodiment is shown in FIGS. 8, 9, and 10. In this embodiment, wires 420, 422, and 434 could extend through the handle 43 of the biopsy device and emerge through the proximal end of the biopsy device. Wires 420, 422, and 434 could be attached to connector 428 and fixed each to one of contacts 433, 435, and 437. Contacts 439, 441, and 443 in groove 430 on the underside of handle 43 would contact contacts 433, 435, and 437 on connector 428 when electrode sleeve 410 is connected to handle 43. FIG. 11 shows the underside of probe assembly 40 and electrode sleeve 410 with electrode sleeve 410 slightly distal to handle 43. Connector 428 is shown with contacts 433, 435, and 437 schematically illustrated at a proximal end of sleeve 410.

FIG. 11a shows groove 430 on the underside of handle 43, and contacts 439, 441, and 443 placed in groove 430. When electrode sleeve 410 abuts handle 43, connector 428 is in groove 430. Contacts 433,435, 437 are in electrical contact with contacts 439, 441, and 443, respectively, to provide for energizing the electrodes.

In another embodiment, the three or more electrodes can be positioned on the sleeve 410. With three or more electrodes, an interface relay circuit board can be used to switch charge among the electrodes in schemes that alternate coagulation to different portions of the tissue in contact in surface with the sleeve or the piercer.

In another embodiment, only one electrode could be utilized in a monopolar arrangement. A grounding pad, placed under the patient as is practiced while using monopolar RF energy, could be utilized while the single electrode is charged. Alternately, the single electrode could be utilized with the knife tip and a bipolar RF arrangement.

In another embodiment, any electrode on the piercer or on the sleeve may have a variety of geometries that efficiently coagulate the tissue. An electrode, for example, may surround at least a portion of the circumference of the sleeve or piercer as would a ring, and have an axial length along the sleeve or piercer.

In another embodiment, a switching cycle utilized by the components illustrated in FIG. 4 by numeral 436 can be programmed so that the switching rate is zero, so that only two electrodes become charged. In this embodiment, a surgeon using the biopsy device may choose to charge two electrodes and may manually change the set of electrodes charged. The set of electrodes being charged can be changed through the program. The surgeon inputs to computer 438 the set of electrodes to be charged. Each time a new set of electrodes is selected, the new set can be input to computer 438.

It will also be recognized by one skilled in the art that some or all of the components identified by reference numeral 436 may be incorporated as an integral part of hardware and software used to control the cutting and suction portions of a process used with a biopsy device. A computer console may also be employed for controlling some or all aspects of cutting, suction, cauterization, and electrode switching.

Referring now to FIGS. 14-20, a method and device are disclosed according to the present invention for obtaining multiple tissue samples, such as for treating benign PBH.

Referring to FIG. 14, a handheld ultrasound device 1000 is shown positioned trans-anally into the rectum 3000 such that the ultrasound probe 1100 is positioned to visualize the prostate 3100. A balloon catheter 1200 may be positioned in the urethra 3200 such that the tip 1205 of the catheter 1200 is disposed in the bladder 3300 and a balloon portion 1210 of the catheter is disposed in a portion of the urethra extending through the prostate 3100. The balloon portion 1210 can be inflated or at least partially inflated to help position and steady the portion of the urethra disposed in the prostate, and to help visualize the urethra under ultrasound.

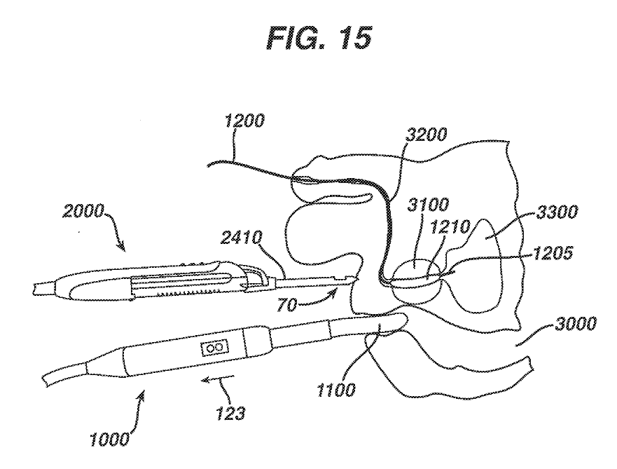

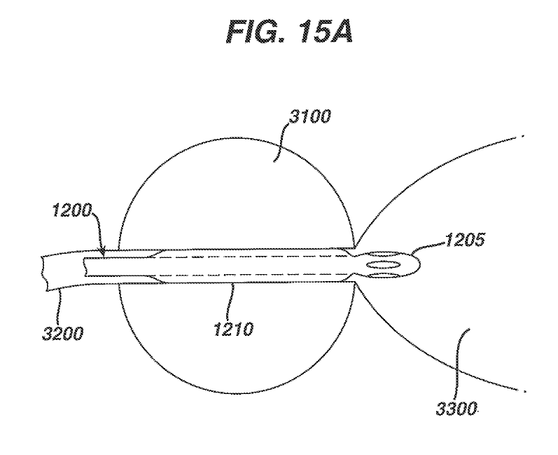

Referring to FIG. 15, the probe 1100 can be retracted, or withdrawn slightly as indicated by the arrow 123 in FIG. 15, so that the ultrasound device 1000 can be employed to visualize and guide the working portion of a vacuum assisted, multiple sample, single insertion biopsy device 2000. The biopsy device 2000 can be a handheld Mammotome brand biopsy device available from Ethicon Endo-Surgery of Cincinnati, Ohio.

As described above with reference to FIG. 1, biopsy device 2000 can include a hollow tissue piercing element, such as a hollow piercer 70. A hollow cutter supported for translation and rotation within a lumen of hollow piercer 70 has a sharpened distal edge for cutting tissue received in a lateral tissue receiving port 78 spaced proximally from the closed distal end of the piercer 70.

Referring again to FIG. 15, the probe 1100 can be used to guide insertion of the piercer 70. Ultrasound probe 1100 can be used to position the port 78 at a desired position within the prostate tissue. Referring to FIG. 15A, the catheter 1200 is shown with catheter tip 1205 disposed in the bladder, and with inflatable balloon portion 1210 of the catheter at least partially inflated to provide support to the portion of the urethra 3200 extending through prostate 3100. The inflated portion 1210 may also assist in ultrasound imaging of the urethra by providing a defined shape that can be identified by remote imaging. For instance, the air space or void provided by the inflated portion 1210 may enhance ultrasound image visualization of the urethra, so that the surgeon may more easily take tissue samples from the prostate while avoiding cutting or otherwise injuring the urethra.

An incision can be made in the anal verge to allow insertion of the piercer into the prostate, or alternatively, the distal tip of the piercer can be employed to insert the piercer through the skin of the anal verge.

If desired, the piercer 70 can be guided by other imaging methods, such as MRI. If desired, the piercer 70 can be formed of a suitable MRI compatible material which is non-magnetic, and can have a generally non-metallic structure, such as by being formed of a ceramic or polymeric material.

An electrode sleeve 2410 can be advanced into the prostate with the piercer 70, or alternatively, the sleeve 2410 can be advanced over the piercer after samples of prostate tissue have been taken. In the description below, the sleeve 2410 is advanced with the piercer 70 into tissue.

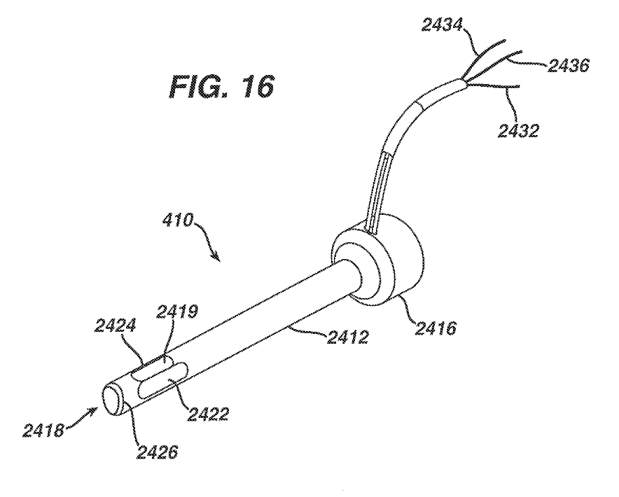

The sleeve 2410 can be generally of the type described above with respect to sleeve 410. In one embodiment, the sleeve 2410 can include three electrodes. Referring to FIG. 16, the sleeve 2410 can include a thin, flexible tube 2412 having an internal lumen sized to receive the piercer 70. The flexible tube 2412 can extend distally from a hub 2416, and the tube can have an open distal end 2418, and a lateral side port 2419 sized and shaped to be registered with the port 78 when the piercer 70 is inserted in the sleeve 2410. The sleeve 2410 can include three electrodes 2422, 2424, and 2426. The electrodes 2422 and 2424 can be associated with opposite sides of the port 2419, while electrode 2426 is shown associated with the distal open end 2418. Electrical leads 2432, 2434, and 2436 can extend from the sleeve hub 2416 and provide current/power to energize electrodes 2422, 2424, and 2426.

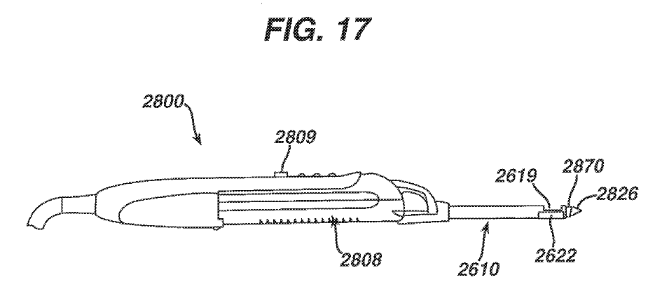

Alternatively, a third electrode can be provided at the distal end of the piercer, such as by being attached to or integral with a distal piercing tip of the piercer. Referring to FIG. 17, for instance, an alternative single insertion, multiple sample vacuum assisted biopsy device 2800 is depicted. The device 2800 includes a piercer 2870 having a conductive distal piercing tip 2826. A sleeve 2610 is shown positioned over the piercer 2870. The sleeve 2610 has a lateral tissue receiving opening 2619, and two electrodes, such as a first electrode 2622 associated with a first longitudinally extending edge of the opening 2619, and a second electrode (not visible in FIG. 17) associated with the opposite longitudinally extending edge of the opening 2619. The sleeve 2610 can be electrically coupled to the handpiece 2808 of the biopsy device 2800, such that the device 2800 can provide energy/electrical current to the electrodes on the sleeve 2610 as well as to the distal tip electrode 2826, such as with one or more control button 2809 on the handpiece 2808 of device 2800.

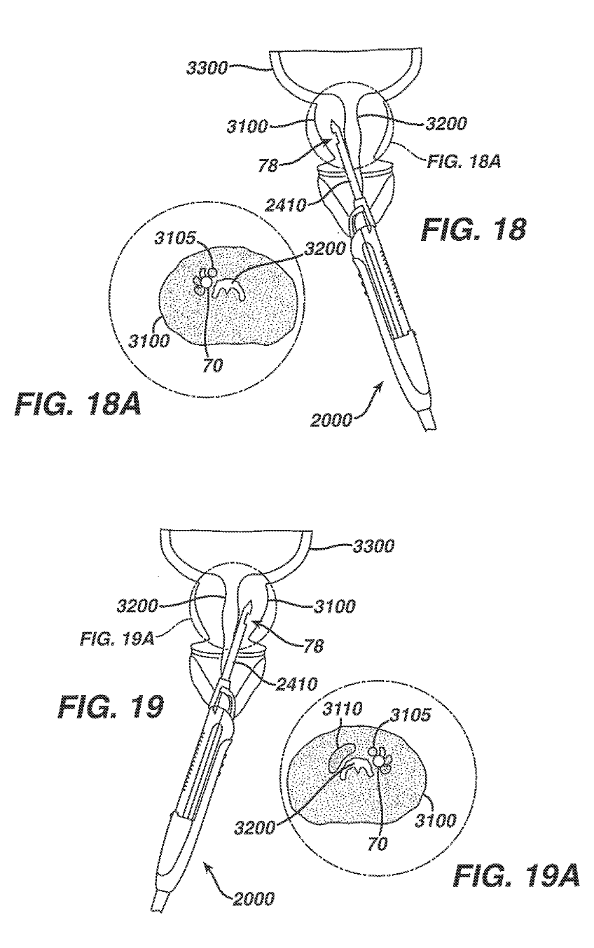

Referring to FIGS. 18 and 19, first and second placements of the piercer 70 are illustrated. In FIG. 18, with the sleeve 2410 (or 2610) disposed on the piercer 70, the piercer is inserted through the anal verge into the prostate on one side of urethra, such at approximately a 9 o'clock position (generally left side of prostate as viewed in FIG. 18) with respect to the urethra 3200. The port 2419 (or 2619) of the sleeve and the port 78 of the piercer are rotated such that they are generally aligned and such that both face away from the urethra 3200, so that prostate tissue samples to be taken through piercer 70 is spaced from urethra 3200 by the piercer 70. Such spacing is illustrated in the cross-section of the prostate shown in FIG. 18A, where four core samples 3105 are shown removed from the prostate on the side of needle 70 generally opposite the urethra 3200. The four samples 3105 taken in FIG. 18A can be taken without removing the piercer 70 from the prostate.

After each sample is taken, the electrodes (such as the three electrodes 2422, 2426, and 2428; or the three electrodes associated with the device in FIG. 17) can be energized to cauterize the tissue site from which the sample is taken. Alternatively, the electrodes can be energized after multiple samples are taken and before and during removal of the needle 70 from the 9 o'clock position in the prostate. The electrodes can be energized in a predetermined pattern, such as by being controlled manually or by a computer or microprocessor, such that electrodes such as 2422 and 2426 are energized or otherwise provide a current path therebetween, then electrodes 2422 and 2428, then electrodes 2426 and 2428. By sequentially providing a current/RF energy between pairs of electrodes in an alternating fashion, the electrode sleeve 2410/2610 can be employed to cauterize the core wall and reduce/eliminate bleeding.

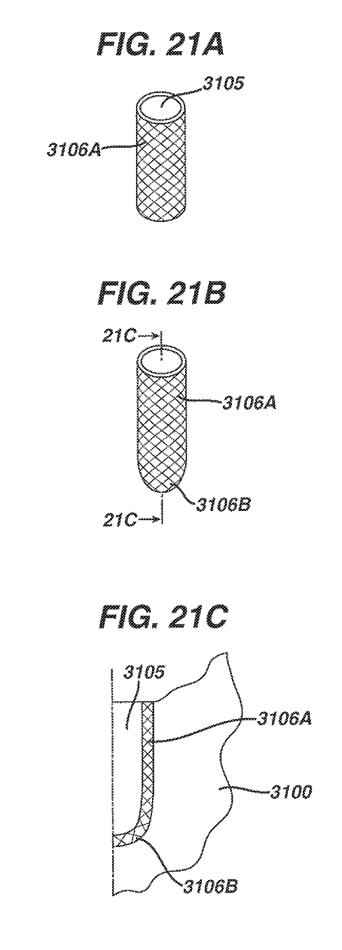

For instance, when the energy path is between the two side electrodes such as 2422 and 2426, the RF energy provided serves to cauterize a generally cylindrically shaped surface portion of the tissue mass from which the sample(s) are taken. FIG. 21A illustrates a generally cylindrical shaped surface portion of a volume of cauterized tissue 3106A such as tissue surrounding a volume from which a core sample 3105 is taken.

When the energy path is between either of the two side electrodes and the end electrode 2426 (or electrode 2826), the RF energy provided serves to coagulate an end portion of the tissue mass from which the sample(s) are taken. FIG. 21B illustrates a volume of tissue including a generally cylindrical volume of cauterized tissue 3106A (as shown in FIG. 21A) with an end cap shaped (e.g dome shaped) volume of cauterized tissue 3106B added at the end of the volume 3106A.

FIG. 21C illustrates a partial cross-section as taken along the lines 21C-21C in FIG. 21B and illustrating cauterized tissue 3106A and 3106B in prostate 3100 adjacent a volume from which a core sample 3105 has been taken. As a result, a generally 360 degree cylindrical volume of cauterized tissue and a distal end volume of cauterized tissue can be provided to prevent or reduce bleeding as the core samples 3105 are taken.

The four core samples 3105 shown in FIG. 18A can be taken by slightly rotating the needle/piercer 70 about its axis to obtain multiple samples closely spaced together, such that a generally oblong void/cavity 3110 is provided. The void/cavity 3110 at the 9 o'clock position is shown schematically in FIG. 19A. After the cavity 3110 is formed at the 9 o'clock position, the sleeve and piercer 70 can be withdrawn from the prostate (but not necessarily completely from the body).

In FIG. 19, with the sleeve disposed on the piercer 70, the piercer is reinserted in the prostate, this time at approximately the 3 o'clock position with respect to the urethra (the right side as viewed in FIG. 18). The ports 78 and 2419 are positioned such that they face away from the urethra, and multiple samples of prostate tissue can be taken through piercer 70 without removing the piercer 70 from the prostate. As shown in FIG. 18A, the multiple core samples 3105 (four shown in FIG. 18A) can be closely spaced together, and spaced from the urethra by the piercer 70.

Referring to the FIGS. 18 and 19, the piercer 70 can be repositioned in different parts of the prostate by removing the piercer from the prostate but not through the skin surface, and angling the piercer to advance the piercer into another portion of the prostate (e.g. different o'clock position with respect to the urethra). Alternatively, the piercer can be inserted along a first insertion path through the skin and into the prostate to take multiple samples from a particular o'clock position. Then, after one or more samples are taken from a first o'clock position in the prostate, the piercer 70 can be fully withdrawn through the outer surface of the skin, and the needle 70 can then be reinserted through the skin and into the prostate along a second insertion path that is substantially parallel to the original insertion path through the skin of the anal verge. Additional punctures through the skin and or prostate may be made as needed to provide access and debuling as needed.

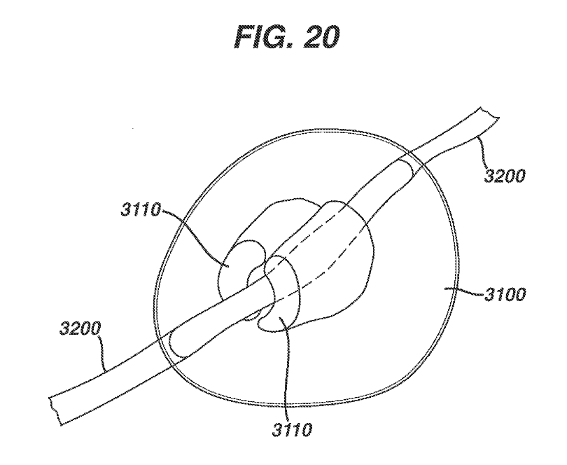

FIG. 20 provides a perspective schematic illustration of the cavities 3110 formed on generally opposite sides of a portion of the urethra 3200 extending through the prostate 3100. The device and method disclosed may be used reduce the size of/debulk an enlarged prostate in a manner that minimizes risk of damage to nerve bundles and/or the portion of the urethra extending through the prostate.

While the present invention has been illustrated by description of several embodiments, it is not the intention of the applicant to restrict or limit the spirit and scope of the appended claims to such detail. Numerous other variations, changes, and substitutions will occur to those skilled in the art without departing from the scope of the invention. For instance, the device and method of the present invention has been illustrated in relation to coagulation of breast tissue, but it will be understood the present invention has applicability in other tissues as well. Moreover, the structure of each element associated with the present invention can be alternatively described as a means for providing the function performed by the element.

It will be understood that the foregoing description is provided by way of example, and that other modifications may occur to those skilled in the art without departing from the scope and spirit of the appended Claims.

What is claimed is:

1. A method of obtaining a tissue sample from the prostate, the method comprising the steps of:
positioning a hollow tissue piercing element having a closed distal end and a tissue receiving port spaced proximally of the distal end into the prostate;
taking a first tissue sample of prostate tissue through the hollow tissue piercing element;
rotating the hollow tissue piercing element within the prostate; and
taking a second tissue sample of prostate tissue through the hollow tissue piercing element.

2. The method of claim 1 comprising the steps of:
positioning the hollow tissue piercing element into the prostate in a first position with respect to one side of the urethra;
taking multiple tissue samples from the prostate adjacent the first position a without removing the hollow tissue piercing element from the prostate;
withdrawing the hollow tissue piercing element from the prostate; and
repositioning the hollow tissue piercing element within the prostate at a second, different position with respect to the urethra; and
taking multiple tissue samples from the prostate adjacent the second position without removing the hollow tissue piercing element from the prostate; and
removing the hollow tissue piercing element from the prostate.

3. The method of claim 1 comprising the step of advancing a cutter within the hollow tissue piercing element to severe a tissue sample.

4. The method of claim 3 comprising advancing and rotating a hollow cutter within the hollow tissue piercing element to severe a tissue sample.

5. The method of claim 1 comprising taking prostate tissue samples on substantially opposite sides of the urethra.

6. The method of claim 5 comprising taking multiple tissue sample through the hollow tissue piercing element at substantially a 3 o'clock position and substantially a 9 o'clock position with respect to the urethra.

7. The method of claim 1 comprising placing an ultrasound probe in the rectum prior to inserting the hollow tissue piercing element into the prostate.

8. The method of claim 7 comprising visualizing the prostate with the ultrasound probe.

9. The method of claim 8 further comprising retracting the ultrasound probe to a position in the rectum to permit ultrasound imaging guided insertion of the hollow tissue piercing element into the prostate.

10. The method of claim 1 further comprising the step of positioning a catheter in the urinary tract prior to taking a tissue sample through the hollow tissue piercing element.

11. The method of claim 10 comprising inflating a balloon in a portion of the urethra prior to taking a tissue sample through the hollow tissue piercing element.

12. The method of claim 1 further comprising the step of applying RF energy to a portion of the prostate associated with tissue sampling.

13. The method of claim 12 comprising cauterizing a portion of the prostate associated with tissue sampling.

14. The method of claim 1 further comprising positioning at least one electrode over the hollow tissue piercing element to delivery RF energy to a portion of the prostate associated with tissue sampling.

15. The method of claim 14 comprising positioning multiple electrodes over the hollow tissue piercing element.

16. The method of claim 1 further comprising positioning a hollow sleeve carrying multiple electrodes over the hollow tissue piercing element.

17. The method of claim 1 comprising
inserting the hollow tissue piercing element along a first insertion path into the prostate;
taking at last one tissue sample of the prostate through the hollow tissue piercing element;
withdrawing the hollow tissue piercing element from the prostate and through outer skin;
reinserting the hollow tissue piercing element through the skin and along a second insertion path different from the first insertion path; and
taking at least one tissue sample of the prostate through the hollow tissue piercing element positioned along the second insertion path.

18. A method of treating benign prostate enlargement, the method comprising the steps of:
positioning an ultrasound probe in the rectum;
positioning a catheter in a portion of the urethra within the prostate;
positioning the hollow tissue piercing element of a single insertion, multiple sample vacuum assisted biopsy device into the prostate under ultrasound guidance;
taking prostate tissue samples through the hollow tissue piercing element at substantially 3 o'clock and substantially 9 o'clock with respect to the portion of the urethra passing through the prostate to provide voids in the prostate; and
advancing a sleeve having a plurality of electrodes over the hollow tissue piercing element and into the prostate; and
activating the electrodes to cauterize at least a portion of the prostate tissue adjacent the voids.

19. A method of removing tissue samples from within a tissue mass comprising the steps of:
obtaining a vacuum assisted single insertion, multiple sample biopsy device;
taking multiple tissue samples from within the tissue mass through the biopsy device;
positioning first, second, and third electrodes with respect to a portion of the tissue mass from which the samples have been taken;
energizing the first electrode and the second electrode while the third electrode is not energized;
energizing the first electrode and the third electrode while the second electrode is not energized; and
energizing the second electrode and the third electrode while the first electrode is not energized.

20. The method of claim 19 wherein at least one of the electrodes is operatively associated with a tissue piercing element of the biopsy device;

21. The method of claim 19 further comprising the step of providing support to the portion of the urethra within the prostate during the step of taking tissue samples.

22. The method of claim 21 wherein the step of providing support comprises at least partially inflating a member within the urethra.
